(19) United States
(12) Patent Application Publication
(10) Pub. No.: US 2016/0113587 A1
(43) Pub. Date: Apr. 28, 2016

(54) ARTIFACT REMOVAL TECHNIQUES WITH SIGNAL RECONSTRUCTION

(71) Applicant: THE REGENTS OF THE UNIVERSITY OF CALIFORNIA, Oakland, CA (US)

(72) Inventors: Christian Andreas Edgar Kothe, San Diego, CA (US); Tzyy-Ping Jung, San Diego, CA (US)

(21) Appl. No.: 14/895,440

(22) PCT Filed: Jun. 3, 2014
(86) PCT No.: PCT/US14/40770

§ 371 Date: Dec. 2, 2015

Related U.S. Application Data
(60) Provisional application No. 61/830,595, filed on Jun. 3, 2013.

(51) Int. Cl. A61B 5/00; A61B 5/0476; A61B 5/0205; A61B 5/0402; A61B 5/0488; A61B 5/04; A61B 5/12

(52) U.S. Cl. CPC A61B 5/7214; A61B 5/04012; A61B 5/0476; A61B 5/04008; A61B 5/125; A61B 5/0402; A61B 5/0488; A61B 5/0205; A61B 5/7253; A61B 5/7264; A61B 2560/0223

(57) ABSTRACT

Methods, systems, and devices are disclosed for removing non-stationary and/or non-stereotypical artifact components from multi-channel signals. In one aspect, a method for processing a signal includes obtaining a first signal decomposition of a multi-channel baseline signal in a first matrix including nominal non-artifact signal components and a second signal decomposition of a multi-channel data signal in a second matrix including artifact components, in which the first and second matrices are complimentary matrices, forming a linear transform by non-linearly combining the complementary matrices, and producing an output signal corresponding to the multi-channel data signal by applying the formed linear transform to one or more samples of the multi-channel data signal to remove artifacts and retain non-artifact signal content in the output signal.

### CROSS-REFERENCE TO RELATED APPLICATIONS

This patent document claims benefit of priority of U.S. Provisional Patent Application No. 61/830,595, entitled “HIGH-AMPLITUDE ARTIFACT REMOVAL WITH SIGNAL RECONSTRUCTION” and filed on Jun. 3, 2013. The entire content of the aforementioned patent application is incorporated by reference as part of the disclosure of this patent document.

### STATEMENT REGARDING FEDERALLY SPONSORED RESEARCH OR DEVELOPMENT

This invention was made with government support under grant W911NF-10-2-0022 awarded by the Army Research Lab (ARL). The government has certain rights in the invention.

### TECHNICAL FIELD

This patent document relates to signal processing technologies.

### BACKGROUND

Signal processing is a discipline of engineering, physics, and applied mathematics that involve a variety of techniques and tools that perform operations on or analysis of signals and/or measurements of time-varying or spatially varying physical quantities. For example, signal processing techniques can be used for signals including sound, electromagnetic radiation, images, and sensor data, e.g., such as biological and physiological data such as electroencephalograms (EEG), magnetoencephalogram (MEG), electrocochleograms (ECochG), electrocorticograms (ECoG), electrocardiograms (ECG), and electromyograms (EMG), among many others.

### SUMMARY

Techniques, systems, and devices are disclosed for removing artifact components (e.g., contaminated or noise components) from a signal by reconstructing the contaminated data of the artifact components with non-contaminated signal content. The disclosed signal processing methods can adequately mark components for removal while selectively preserving the portion of activity in the removed component that is not contaminated. In some implementations, non-stationary and/or non-stereotypical high-amplitude “artifact” (e.g., contaminated) components can be removed from multi-channel signals, in which contaminated data are reconstructed from non-contaminated signal contents.

The subject matter described in this patent document can be implemented in specific ways that provide one or more of the following features. For example, the disclosed technology can provide the ability to robustly remove most types of high-amplitude, non-stationary and non-stereotypical artifacts from ongoing physiological signals, e.g., such as EEG, ECoG, MEG, ECG with very low (and frequently negligible) residual. This can in turn provide the ability to transfer a vast array of signal processing techniques that are known to be prone to artifacts to circumstances where artifacts cannot be avoided, e.g., such as real-time signal processing during outdoor activities (e.g., recreational), at workplaces where frequent movement and use of head or face musculature occurs (e.g., industrial operators, call centers), military operations (e.g., environments with strong shaking, acceleration and rapid movement), or for video game applications where considerable head/face movement occurs. In some implementations, for example, the disclosed technology can be applied to online processing of data, e.g., including incremental processing, causal processing, and real-time processing, as well as offline processing of data, e.g., including batch processing.

Those and other features are described in greater detail in the drawings, the description and the claims.

### BRIEF DESCRIPTION OF THE DRAWINGS

FIG. 1A shows a diagram of an exemplary signal processing method of the disclosed technology.

FIG. 1B shows a block diagram of the exemplary signal processing method of FIG. 1A.

FIG. 2 shows a data plot depicting exemplary results of exemplary implementations of the disclosed method.

FIG. 3 shows a diagram of an exemplary processing unit configured to implement the disclosed signal processing methods.

FIG. 4 shows a diagram of an exemplary system of the disclosed technology including a real-time data processing pipeline.

FIG. 5 shows a data plot depicting exemplary results of exemplary implementations of the disclosed technology.

FIG. 6 shows a diagram depicting an exemplary temporal snapshot of reconstructed source networks of the exemplary results.

FIG. 7 shows a data plot depicting exemplary results of exemplary implementations of the disclosed technology.

FIG. 8 shows an ERP image depicting exemplary results of exemplary implementations of the disclosed technology.

### DETAILED DESCRIPTION

Virtually all signals include noise or errors that are may cause undesirable modifications or artifacts to the signal. Signal artifacts may be caused during the creation or capture, storage, transmission, processing, and/or conversion of the signal. For example, in electrophysiological monitoring, artifacts include anomalies such as interfering signals that originate from some source other than the electrophysiological structure or event of interest. Examples of such anomalies include signals generated from energy sources, internal signals within instrumentation utilized in measurement, a utility frequency (e.g., 50 Hz and 60 Hz), and/or interfering electrophysiological signals from that of interest. Artifacts may obscure, distort, or completely misrepresent the true underlying signal sought.

Existing methods for artifact removal in electrophysiological multi-channel time series can be characterized as channel-based or component-based. Channel-based methods operate on a channel-by-channel basis, e.g., by interpolating a contaminated channel. Component-based methods represent the signal as a sum of components (some of which may be artifacts), which assumes the existence of a collection of latent source components, each of which linearly projects onto typically multiple channels and which together additively comprise the observed signal (optionally plus random noise, e.g., Gaussian-distributed). Existing component-based methods either assume stationary source projections onto channels or non-stationary projections. For example, most existing component-based methods assume stationary projections due to the relative difficulty of estimating the required coefficients in a time-varying manner on short periods of data. One existing non-stationary method utilizes a short-time Principal Component Analysis (PCA) of the signal to identify high-amplitude components.

Other conventional methods for artifact removal include template-based methods, regression-based techniques, and wavelet-based methods. Template-based methods assume that only a tractably small number of stereotypical cases of artifacts occur, which can be identified based on template matching (e.g., such as eye-blink patterns in EEG). Template-based methods cannot handle artifacts that are random noise processes or where the number of unique cases is intractable, e.g., such as methods based on machine learning of templates. Regression-based techniques, e.g., including most adaptive-filter based techniques, require a reference signal that contains a (more or less) pure realization of the artifact itself. One limitation of regression-based techniques lies in that a practical reference signal does not exist in general. Wavelet-based methods assume that the artifacts are temporally structured such that they can be captured by a small number of Wavelet coefficients. However, the methodology behind wavelet-based methods does not apply to unstructured or quasi-random artifacts, e.g., such as muscle artifacts in EEG. Also, for example, the same limitation applies to other types of temporal model-based methods, e.g., such as assuming the artifact to be comprised of damped sinusoidals.

Existing techniques associated with removal of non-stereotypical artifacts from signals including multi-channel time series, e.g., such as EEG, ECoG, MEG, ECG signals, are typically component-based techniques. What is common to essentially all existing component-based artifact removal methods is that only negligible statistical relationships between a signal's constituent components are assumed, e.g., since the decompositions giving rise to the components either assume uncorrelatedness (e.g., for Principal Component Analysis based techniques) or statistical independence (e.g., for Independent Component Analysis based techniques), or sparse and typically undercomplete activations (for sparse estimation based techniques). As a consequence, once a component has been selected for removal, there is no principled way to reconstruct the original (uncontaminated) signal portion of the component itself, since all remaining components are by design assumed to be unrelated and therefore do not help explain the missing signal content.

For this reason, essentially any existing component-based artifact removal method sets the component's activation to zero, or equivalently subtracts the content of the component, or equivalently reconstructs” the signal from all other components, and therefore (i) loses signal energy and (ii) results in pathological signal content for subspaces of interest, e.g., thereby creating a major issue for subsequent signal processing and analysis stages.

Disclosed are methods, systems, and devices for processing a signal containing information.

In some aspects, methods are disclosed for removing artifact components (e.g., contaminated or noise components) from a signal by reconstructing the contaminated data of the artifact components with non-contaminated signal content. The disclosed signal processing methods can adequately mark components for removal while selectively preserving the portion of activity in the removed component that is not contaminated.

In some implementations, the artifact components removed by the disclosed methods can include high-amplitude, non-stationary and/or non-stereotypical artifacts from ongoing signals in real-time. For example, the disclosed methods can be applied to physiological signals, e.g., such as EEG, ECoG, MEG, ECG, and EMG, among others, e.g., with very low (and frequently negligible) residual. In some implementations of the disclosed methods, the signals can include multi-channel signals, or single channel signals that have been expanded to a multi-channel signal, e.g., by using techniques such as delay-embedding, where lagged versions of the same channel are taken as multiple channels, and/or by other techniques, possibly non-linear, for generating the multi-channel signal, e.g., via a kernel embedding.

### Exemplary Embodiments

In an exemplary embodiment of the disclosed signal processing method, the method includes using two complementary signal decompositions (e.g., component representations), in which one serves as a baseline that characterizes the nominal (non-corrupted) signal content (e.g., the baseline decomposition), and the other characterizes the potentially corrupted current (short-time) signal content under consideration for repair (e.g., the current decomposition). Each signal decomposition is a complete representation, e.g., defining a full-rank matrix. In some implementations, the two decompositions can be estimated given different portions of the signal, e.g., from a baseline window in the case of the baseline decomposition, and from the current window in the case of the current decomposition, and/or the two decompositions can be estimated utilizing different and complementary statistical assumptions, e.g., such as artifact-sensitive statistics on the current signal window vs. statistics for estimating nominal signal parameters on a baseline signal window. The two decompositions are used by a reconstruction method, e.g., in which the current signal can be assumed to be a sum of nominal contents plus potentially some artifact contents, so that both decompositions explain the same data in an over-determined manner.

For example, the reconstruction logic of the disclosed signal processing method can include first partitioning the components of the current decomposition into artifact and non-artifact components using a threshold-like criterion that is sensitive to a quantifiable characteristic of the signal (e.g., such as the amplitude or variance of the signal, a time course or pattern such as a sinusoid or ECG pattern, or other characteristic) in the signal component that it is applied to. And second, for example, the reconstruction logic of the disclosed signal processing method can include applying a linear transform that re-estimates the content of the artifact subspace from the non-artifact subspace in the current signal, e.g., since two complete representations are available that describe the signal in an overcomplete manner. For example, this process can involve a non-linear operation that amounts to the equivalent of solving an underdetermined linear system that designs a linear transform (given the two decompositions). The linear transform can be applied to a set of samples in a time window with the effect of significantly removing the artifact content in the output, e.g., optionally with adequate tapering for overlap-add online processing.

The current decomposition and the baseline decomposition of the disclosed signal processing method are computed and supplied to the reconstruction logic by separate “feeder” stages, where the current decomposition is dependent on the current signal segment.

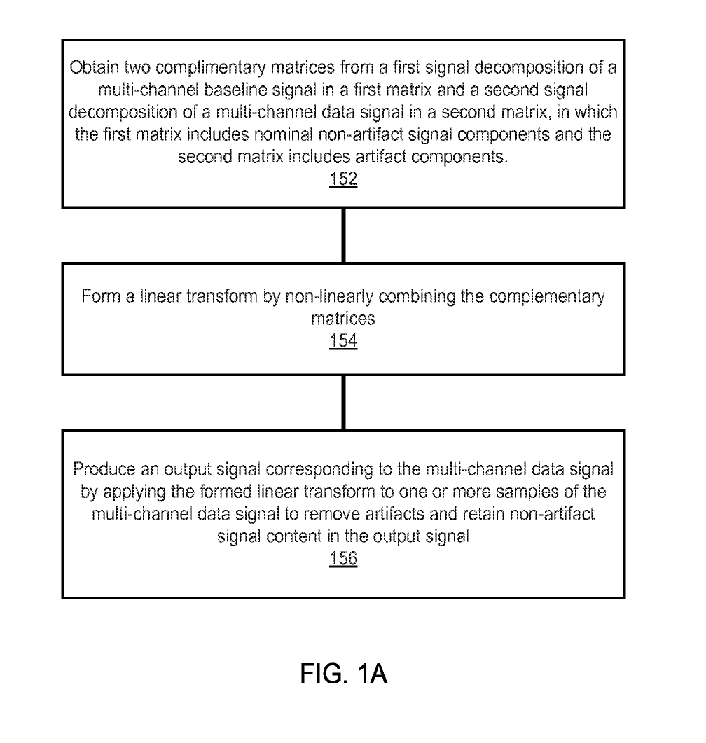

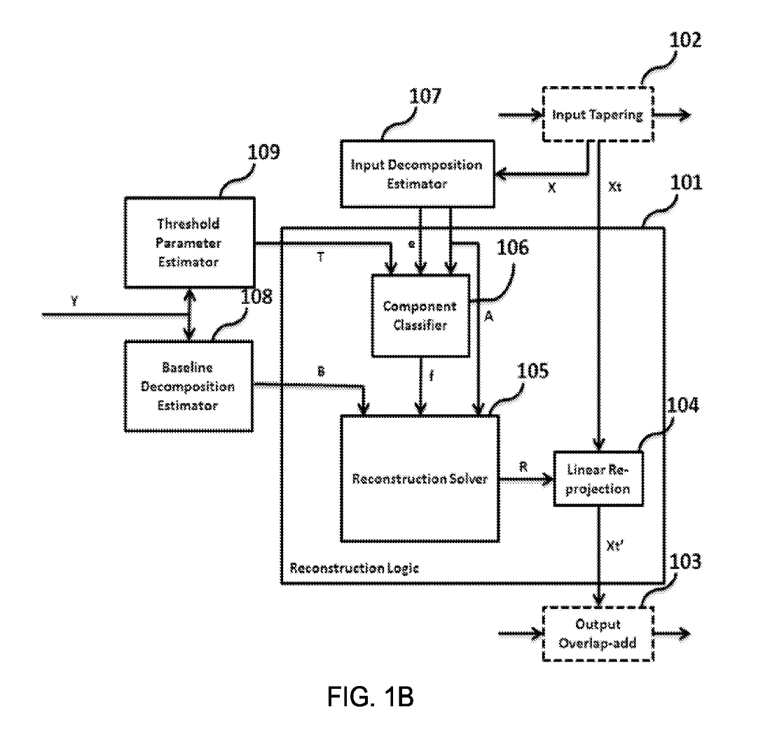

FIG. 1A shows a diagram of an exemplary signal processing method of the disclosed technology to remove artifacts from multi-channel signals. The method includes a process 152 to obtain a first signal decomposition of a multi-channel baseline signal in a first matrix including nominal non-artifact signal components and to obtain a second signal decomposition of a multi-channel data signal in a second matrix including artifact components, in which the first and second matrices are complimentary matrices. The method includes a process 154 to form a linear transform by non-linearly combining the complementary matrices. The method includes a process 156 to produce an output signal corresponding to the multi-channel data signal by applying the formed linear transform to one or more samples of the multi-channel data signal to remove artifacts and retain non-artifact signal content in the output signal.

Implementations of the method can include one or more of the following exemplary features. In some implementations, the process to form the linear transform by non-linearly combining the two complementary matrices can include classifying the signal components into artifact and non-artifact components using a binary classification criterion. For example, the binary classification criterion can be selected as a quantifiable characteristic of the signal sensitive to the amplitude, variance, and/or a pattern of the time course of the signal. For example, the binary classification criterion can include a data-dependent or empirically estimated parameter or parameters, in which the parameter or parameters provide a soft or hard threshold to classify the signal components. Examples of providing a soft threshold include using a sigmoidal response function, and examples of providing a hard threshold include using a step function. In some implementations of the method, for example, the multi-channel signal can include an EEG, MEG, ECoG, ECochG, ECG, and/or EMG signal. In some implementations, for example, the multi-channel signal can include a single-channel signal augmented to multiple channels, in which at least some channels of the multiple channels have a replicate of the single-channel signal that is temporally modified.

In some implementations of the method, for example, when the artifact content in the multi-channel data signal (e.g., the to-be-cleaned or corrected signal) is assumed to be non-stationary (e.g., based on non-stationary projections/changing sets of components, etc.), then the signal decomposition of the multi-channel data signal can be applied to one or more segments of that data (e.g., otherwise overlapped segmentation is not strictly necessary). In some implementations, for example, the baseline signal can include a multi-channel calibration signal (e.g., containing presumably no artifacts) corresponding to the multi-channel data signal, which may contain artifacts to be corrected. In some implementations, for example, the multi-channel baseline signal includes at least a portion of the multi-channel data signal. For example, the baseline signal used in the method can be the same signal as the data signal used. In such implementations, for example, the method can include using an estimator to form the first matrix (e.g., of the nominal, non-artifact signal components) that is insensitive to robust artifacts.

In some implementations of the method, for example, the multi-channel baseline signal can include a calibration signal corresponding to the multi-channel data signal or a predetermined signal. In some implementations, for example, the first signal decomposition can be configured as a component representation of the multi-channel baseline signal. In some implementations, for example, the second signal decomposition can be configured as a component representation of the multi-channel data signal, or as a component representation of the multi-channel baseline signal.

In some implementations of the method, for example, the second matrix can include eigenvectors of a principal component analysis of a short signal segment or resulting from an alternative decomposition of the short signal segment capable of isolating high-amplitude components, and in which the non-linearly combining the two complementary matrices can include solving an underdetermined linear system. In some implementations, for example, the method can further include estimating the second matrix from data containing low artifact content, the estimating including making an explicit or an implicit assumption of low artifact content.

FIG. 1B shows a block diagram of an exemplary signal processing method of the disclosed technology. For example, the signal processing method includes a signal reconstruction method, which is depicted by the reconstruction logic (block 101) at the core of the signal processing method. The reconstruction logic block 101 includes the pathway of an input signal Xt, which can be the entirety of the multi-channel signal to be processed, or a segment of that signal, possibly overlapped with previous and/or subsequent segments. The input signal Xt can be provided from an optional initial taper stage (block 102) to the reconstruction logic block 101. For example, in implementations using the taper stage block 102, a window function or taper function can be applied to the signal, that is, reweights the segment such that there is a smooth falloff to zero amplitude at both ends of the window. An input signal X that is used for statistical analysis of the immediate neighborhood of Xt can be provided from the optional taper stage block 102 to one or more various blocks, e.g., such as an input decomposition estimator (block 107), that feeds information to the reconstruction logic block 101. The signal processing method includes a baseline decomposition estimator (block 108) that provides the baseline decomposition matrix B to the reconstruction logic block 101; the baseline decomposition estimator may utilize a baseline signal Y in order to determine the baseline decomposition. For example, the baseline decomposition can include a matrix containing the nominal (non-artifact) signal components. The signal processing method includes a threshold parameter estimator (block 109) that provides the matrix T to the reconstruction logic block 101, which represents a quantifiable characteristic of each of the components, e.g., which can be used by the reconstruction logic block 101 as a threshold in the reconstruction method. The reconstruction method (the reconstruction logic block 101) includes a component classifier (block 106), a reconstruction solver (block 105), and a linear re-projection stage (block 104).

The central element of the signal processing method is the reconstruction logic (block 101). The reconstruction logic block 101 receives a zero-mean (e.g., appropriately high-pass filtered) input signal segment Xt which may optionally be tapered (block 102, for example via a Hann window function). The reconstruction logic block 101 outputs a processed version of the signal segment Xt′ from which artifact components (e.g., such as high-amplitude artifact components) have been removed. In some implementations, for example, the output segment Xt′ may be added to an output time series within an overall incremental or real-time signal processing implementation by an optional overlap-add method (block 103). The signal processing method can also be applied to each sample of a time series individually (although at considerable computational cost), and successive tapers and window sizes need not necessarily be identical. The transformation that the reconstruction logic applies to Xt may depend on several other (data-dependent) inputs to the reconstruction logic, as described later in this patent document.

Described here is the operation of the reconstruction logic (block 101). The final stage of this block is a linear transformation (block 104) of the input signal Xt into the output signal Xt′ by applying a re-projection matrix R. The re-projection matrix R is a linear transform formed by the reconstruction solver (block 105) by non-linearly combining two complimentary matrices of signal components, e.g., the matrix B containing nominal or non-artifact signal components, and a matrix A containing artifact components of a short signal segment. Application of the matrix R to the signal serves to ensure that that artifact components of a particular quantifiable characteristic are projected out of the resultant signal. The matrix R is adaptively formed by the reconstruction solver (block 105).

The reconstruction solver (block 105) generates (e.g., estimates) the re-projection matrix R by solving an underdetermined linear system which is given by several other data-dependent matrices including the baseline decomposition (matrix B) and the artifact decomposition (matrix A) by performing a binary classification.:

(1) The artifact decomposition A, which is a full-rank matrix of component vectors that describe components of the current input signal Xt. Each vector in A describes the linear projection of a source component in Xt onto the channels, and it is assumed that, if Xt contains artifact components, these would be representable by a linear combination of a small number of components in A (i.e., A contains candidates of artifact components aside from potentially other signal components).

(2) The baseline decomposition B, which is a full-rank matrix of component vectors which are assumed to describe “nominal” (non-corrupted) signal components in Xt, or at least capture the nominal covariance structure between some latent components and channels in the signal. While both matrices are complete representations of components in Xt, they describe different (and crucially, non-identical) components, namely potential artifacts plus miscellaneous components in the case of A and primarily uncorrupted components in the case of B.

(3) The 0/1-valued flag vector f, which describes which of the components in A are considered artifacts and which are considered non-artifacts. In some implementations, f is a binary input into the reconstruction solver (block 105). For example, without loss of generality in the subsequent description, it is assumed that if the k'th component in A is flagged as an artifact, the k'th element of f is 1, otherwise 0.

The operation of the reconstruction solver (block 105) is to design a matrix such that the component subspace spanned by the artifact components in A is re-estimated from the non-artifact subspace in A, based on the mutual correlation between these two subspaces. While according to A alone there is not necessarily any such correlation, the non-artifact components in B however project into both subspaces (except in the rare circumstance where A's artifact components are perfectly aligned with B's components). In this sense B couples the two subspaces statistically and allows to infer or re-estimate activity in the contaminated subspace from non-contaminated data. Note that, while channels in Xt′ may end up being linearly dependent after an artifact was removed this does not in general reduce the rank of the overall time series, since the applied re-projection matrix varies over time when derived in a segment-by-segment fashion, in particular when artifacts are non-stationary.

To formally derive the operation of the solver, let G=A^{T}B be the baseline decomposition B after transformation into the component space defined by A (instead of the matrix transpose A^{T} one may use the inverse A⁻¹ if A is non-orthogonal throughout the remainder of the description). This matrix describes how components in B project into the components of A. Let H=diag(1−f)G be the restriction of this projection to only the non-artifact subspace in A where diag(1−f) is a diagonal matrix that scales the contribution of B to flagged artifact components to zero. Now define U=H⁺ to be the Moore-Penrose pseudoinverse of H, which is the smallest matrix that solves the problem of estimating the activations of each component in B from only the non-artifact components in A. The overall reconstruction operation is now the concatenation of the following four linear operations: R=AGUA^{T}, which can be read (from right to left) as: 1) project signal into the space of components in A, 2) estimate activations of the baseline components from only the non-artifact components in A, 3) back-project from cleanly estimated baseline components onto all components in A including former artifact components, 4) back-project from the re-estimated components in A onto channels. This operation can be further simplified to the final re-projection matrix R as:

$$\begin{matrix}R = {AA}^{T} {B ( diag ( 1 - f ) {A}^{T} B )}^{+} {A}^{T} \\ = {B ( diag ( 1 - f ) {A}^{T} B )}^{+} {A}^{T}\end{matrix}$$

This equation admits several reformulations that yield equivalent results (for example explicitly splitting the matrix A, or applying the pseudoinverse to a horizontal or vertical concatenation of matrices, or utilizing the singular value decomposition or eigenvalue decompositions to implement the pseudoinverse operator) and some reformulations that yield very similar though neither identical nor more accurate results and it is understood that alternative formulations that result in such solutions for the matrix R and that can be represented in terms of matrices matching the description of A and B are covered within the spirit and scope of the disclosed technology.

Note that it is possible to write the operation diag(1−f)A^{T} for orthonormal A as (Adiag(1−f))⁺, which visually resembles a key term in R, but note that this is merely an expensive way to calculate the transpose of a thresholded A—the simple identity breaks down once the (generally non-orthogonal) matrix B is introduced.

Described here is how the artifact flagging vector f is determined using the component classifier (block 106). The component classifier 106 receives the set of components of matrix A, some of which may be artifacts, as well as an estimate of the per-component energy or amplitude e. For notational clarity, written here is the function applied by 106 to each component v and its associated energy value e as f(v,e) where it is understood that the method is applied in the same manner to all components of A. First and foremost, there is a variety of ways to flag a component v as artifact or non-artifact. The purpose of the method as described is to remove high-amplitude artifacts since these have the greatest (detrimental) effect on the outcome of subsequent signal processing stages (although it is certainly possible to apply the processing also to other kinds of artifact/non-artifact classifications). In line with this goal the criterion is based on the amplitude (or energy) e (or a function thereof) for a given component v, which form the two variable inputs to the classifier. The third input is a threshold representation T, which captures the energy threshold above which a component would be flagged as an artifact. Depending on the exact criterion, T might be a simple scalar threshold for e (such that f(v,e):=1 if e>T, otherwise 0), or a vector that holds per-channel thresholds (then a practical example would be f(v,e):=1 if êa>Tv^{T}, otherwise 0), or a matrix that holds a direction-dependent threshold.

For example, an exemplary embodiment of the component classifier is T being a matrix capturing a direction-dependent threshold where f is defined by f(v,e):=1 if êa>∥Tv∥ₚ, otherwise 0, for some constants a and p (if e is the signal variance in component v, a would ideally be ½ and p would be 2). In this case T would be the concatenation of a transformation followed by a non-uniform scaling, which can be understood as the mapping into a space of components followed by a per-component scalar threshold, followed by the Euclidean norm of the per-component thresholds.

In an alternative embodiment, for example, the method can include defining f(v,e):=1 if e>g(diag(vTv^{T})) otherwise 0 where g( ) is some scalar function (typically monotonic) and T is a covariance matrix, where the term diag(vTv^{T}) represents the projected variance if v is the spatial filter for a given component. The benefit of such a representation is that the free parameter is an easily computed property (as opposed to some component representation with componentwise thresholds) although at the cost of a potentially weaker overall criterion. A more or less principled functional form for g( ) can be derived from X² statistics as they apply to the projected variance.

In some embodiments, for example, the method can implement f(v,e) using a classifier determined via machine learning (for example, a kernel support vector machine) that is parameterized by weights T.

In the following, described here is how the artifact decomposition A for the input signal X can be estimated using the input decomposition estimator (block 107). In some implementations, for example, like that shown in FIG. 1B, the only input to this block is the input signal segment X, possibly tapered, but not necessarily in the same way as Xt (X might cover a longer signal portion, for example). The task of this block is to produce a full-rank matrix of component vectors A such that, if Xt contains artifact components, these can be represented by a small number of components in A (with high probability). There are several ways to arrive at such a matrix. For example, in some implementations, the primary goal may include the capture high-amplitude components accurately. Thus, in one exemplary embodiment, for example, this block is implemented using a Principal Component Analysis (PCA), i.e., an eigenvector decomposition A=eig(cov(X′)) of a signal segment X′ which is either identical to X or a temporally or spectrally filtered/shaped version of X. Another reason for the choice of a PCA are the relatively low computational requirements compared to some other methods, such as Independent Component Analysis, sparse coding, dictionary learning, or variants thereof, which are however included in the spirit of the disclosed technology. The optional filtering, which may be implemented in a variety of ways, such as via a FIR, IIR or FFT-based filter allows to make the sensitivity of the measure A frequency-specific, which allows to incorporate prior knowledge on the spectrum of likely artifact components or conversely non-artifact components into the design of A. Note that this filtered signal X′ does not enter the reconstruction logic block—it only affects the matrix A.

In an alternative embodiment, for example, the decomposition matrix A can be estimated under consideration of some prior knowledge of expected signal components (and potentially independently of X′, although it can be believed that this would severely limit the utility of the method), and in particular knowledge of expected artifact and non-artifact components. In the case of EEG, this can be implemented based on anatomical knowledge of locations and orientations of artifact and non-artifact generators in and around the brain in relationship to the sensor (channel) locations.

Aside from A, the input decomposition estimator can estimate also the energies (or functions thereof) of the components in A as they occur in the signal segment X′ (defined as before). This exemplary arrangement is for simplicity of the exposition only and not intended to imply a limitation of the disclosed technology; without loss of generality these same quantities can also be estimated at any other stage of a practical implementation where both A and X′ (or equivalent) are available. One way to compute these energies is to calculate the variance of the signal after projection by A, i.e., e=var[AX′], or equivalently e=diag(AX′X′^{T}A′^{T}) for a zero-mean signal X′. If A is overcomplete the energies may also be estimated using a more complex estimation procedure, such as the (group) LASSO or sparse Bayesian learning, although it can be assumed this to be a rather expensive approach. An exemplary embodiment of this process, for example, is to obtain the variances of the signal components directly from the eigenvector decomposition of the PCA (namely by defining the vector e as the eigenvalues associated with the components in A).

Described here is the baseline decomposition estimator (block 108). This block produces a full-rank matrix B of latent “nominal” (non-artifact) signal components into which the input signal X may be decomposed. Since it can be assumed that X may both be contaminated and also rather short (perhaps at the limit of statistical sufficiency), X alone may not necessarily be the best piece of data to estimate such a component representation. Nevertheless this is in principle possible (for example using robust estimators like the geometric median covariance matrix) and would be a corner case of the disclosed technology.

There exist several ways to obtain a usable decomposition into nominal components for X since in practice B does not necessarily have to be of the highest possible quality. One particular way is to estimate the decomposition based on prior assumptions about non-artifact components. For example, in case of EEG, this can be implemented based on physical knowledge of source locations, their orientations and the location of the sensors (channels) relative to those sources, for example using an electrophysiological head model (e.g., from magnetic resonance scans). Another is to estimate the components from data in a similar manner as done for the decompositionmatrix A.

In an exemplary embodiment of the disclosed technology, B can be estimated from a signal segment Y, which may either stem from a reference measurement made prior to application of the method, or from a moving signal window typically longer than X that precedes and possibly overlaps X, or, in offline processing, from a potentially large subset of the overall time series to be analyzed. Even if the data in Y can be controlled well enough to ensure low artifact content, for example, it can be preferable to estimate the decomposition in a manner that is robust to artifacts and therefore less affected by their presence. An empirical decomposition may be obtained by using a variety of methods, such as Principal Component Analysis, Independent Component Analysis, sparse coding or dictionary learning, and variants thereof.

An exemplary approach is to first calculating the spatial covariance matrix C of Y in a robust manner. This can be achieved using the geometric median, i.e., optimizing C to be matrix for which the sum of absolute differences between C and YᵢYᵢ^{T} over all samples Y, is minimal. This yields a convex optimization problem that can be solved conveniently using subgradient descent or Weiszfeld's algorithm. Next, B is derived from C as the matrix square root B=sqrtm(C), which can be calculated via an eigenvector decomposition, followed by taking the square root of the eigenvalues, and then re-combining the eigenvectors and eigenvalues.

For example, an alternative embodiment is to replace the geometric median by the regular mean, which yields a non-robust solution that should therefore be derived from non-contaminated data. There are several more exemplary embodiments, listed here for completeness, for example, those that calculate multiple full-rank covariance matrices on short windows and then take a geometric median in a non-Euclidean space using an appropriate distance metric. The drawback of these methods is that they need to pre-average over several samples to arrive at full-rank matrices, which can easily result in artifactual samples entering the computation in ways preventing them from being adequately factored out by the robust estimation part.

Described is the threshold parameter estimation (block 109). This block serves to determine the artifact threshold representation T which is used in the component classifier (block 106) described before. Depending on the type of classifier chosen in an implementation, different estimation procedures are applicable. For example, in general the threshold may be based on prior knowledge alone without considering any available data, for example in case of EEG the threshold might be set at a standard cutoff for EEG voltage, such as 250 mV. In practice a uniform threshold is a rather inadequate choice since not only can the nominal amplitude vary across channels, but it can also depend on the task or other conditions. Nevertheless, using a scalar threshold would be crude application of the disclosed technology. It is also possible to utilize a more sophisticated spatially varying or direction-dependent threshold purely derived from prior knowledge.

In an exemplary embodiment, the threshold parameter T is estimated empirically from a baseline signal, such as the signal Y that is also used to derive the baseline decomposition, although it is possible to utilize a different (potentially more appropriate) signal for this purpose.

In an exemplary embodiment, assumed is that T is estimated as a threshold matrix which is applied in the component classifier in a form similar to ∥Tv∥ₚ (block 106, c.f., previous description of the component classifier). Then, T may be understood as the concatenation of the transformation of a direction vector into a latent component space here denoted as L followed by componentwise non-uniform scaling S=diag(s) for some scales s, such that T=SL^{T}. The vector of scales s represents the thresholds for each component.

Latent component L may be obtained through any mechanism appropriate for learning the baseline decomposition B, and in fact L may be identical to B (in an exemplary implementation it is in fact identical, although this need not necessarily be so).

Given L, the thresholds s for each component may now, for example, be estimated empirically from the matrix of component activations V=L^{T}Y′ where Y′ is either identical to Y or equals Y after application of temporal or spectral filtering of a similar nature as done for X′ in the application of the input decomposition estimator (block 107, c.f. previous description of the input decomposition estimator). Since the thresholds are ultimately to be compared with the energies estimated by block 107, it can be preferable to use filter procedures with the same parameters in both cases. One way to obtain a threshold sₖ for the k'th channel Vₖ (of component activations in V) is to set it to a particular quantile in the empirical distribution of signal energy values observed in Vₖ (either taken over average energy estimates in short successive segments of Vₖ or taken over the single-sample limits, e.g., Vₖ(t)²). One example would be to set them to the 90% quantile. For example, a somewhat more sophisticated way is to estimate not only the mean energy or amplitude in Vₖ but also its variation (i.e., standard deviation), e.g., both in a robust manner (e.g., based on median absolute deviations), and then to set the threshold at z standard deviations above the mean channel energy, where z is a user-configurable parameter (such as z=3). The benefit of this approach is the very reliable behavior and the intuitive semantics of the user-tunable parameter.

An alternative embodiment, for example, is the case where the component classifier is defined in terms of a covariance matrix T as opposed to a threshold matrix (c.f., description of the component classifier). In this case, the estimation procedure for T is simpler and amounts to merely computing (or assuming based on some prior knowledge) a covariance matrix that is characteristic of a nominal signal. Since the covariance matrix needs to be reflective of any temporal or spectral filtering applied in the input decomposition estimator (block 107, cf. previous description), it can be preferably calculated on a signal Y′ after application of temporal or spectral filtering with the same parameters as the one applied to X′ in block 107. Note that a signal different from the baseline Y can be used here as data to be filtered, although an exemplary approach is to use the baseline signal. Given the filtered signal, the covariance matrix T can then be computed either non-robustly as T=cov(Y′), or using a robust method such as the geometric median as outlined in the description of the baseline decomposition estimator (block 108, c.f. previous description). One exemplary way is to use the robust variant.

Since the method is in practice often applied to overlapped segments of data, and therefore to overlapped segments X, as well as possibly to overlapped segments Y (if Y is a running baseline), several efficiency considerations apply, most of which are reformulations of the methods described before with little to no impact on the behavior of the implementation or the mathematics and statistics of the approach.

For clarity of the description, many of these applicable optimizations have been omitted and it is understood that anyone skilled in the relevant arts will be able to derive and apply a variety of well-known reformulations. Such optimizations include, for example, the use of recursive estimation for the relevant statistics (such as recursive moving-average estimation for the various average statistics such as the covariance matrices, recursive running medians for robust statistics, recursive estimation of the geometric median and other non-linear statistics via stochastic (sub-)gradient descent or variants thereof, warm-starting for certain iterative estimation procedures (such as the eigenvalue and singular value decompositions) as well as recursive least-squares techniques or other well-known online variants for computations such as Principal or Independent Component Analysis, dictionary learning and sparse coding). Note that despite a large potential for further optimization of the method, the core implementation is fast enough for practical use in a wide variety of settings, in particular when a fixed baseline window Y is used.

In one exemplary embodiment, the signal processing method to reconstruct a signal removing artifacts can be represented by the exemplary program code below.

For example, the following portion of the program code can be used for implementing blocks 108 and 109, e.g., which can be implemented in MATLAB.

function state=calibrate(X,cutoff,blocksize,B,A)
[C,S] = size(X);
[X,Zf] = filter(B,A,double(X),[ ],2);X = X′;
U = zeros(length(1:blocksize:S),C*C);
for k=1:blocksize

range = min(S,k:blocksize:(S+k−1));
U = U + reshape(bsxfun(@times,reshape(X(range,:),[ ],1,C),...

reshape(X(range,:),[ ],C,1)),size(U));

end
M = sqrtm(real(reshape(block_geometric_median(U/blocksize),C,C)));
[V,D] = eig(M);
X = abs(X*V);
T = diag(median(X) + cutoff*1.3652*mad(X,1))*V′;
state = struct(‘M’,M,‘T’,T,‘B’,B,‘A’,A,‘cov’,[ ],‘carry’,[ ],‘iir’,Zf);

For example, the following portion of the program code can be used for implementing the remaining blocks of FIG. 1B, e.g., which can be implemented in MATLAB.

function
[data,state]=process(data,srate,state,wndlen,lookahead,stepsize,maxdims,maxinem)
[C,S] = size(data);
N = round(wndlen*srate);
P = round(lookahead*srate);
[T,M,A,B] = deal(state.T,state.M,state.A,state.B);
data = [state.carry data];
splits = ceil((C*C*S*8*8 + C*C*8*S/stepsize + C*S*8*2 + S*8*5) / ...

(maxmem*1024*1024 − C*C*P*8*3));

for i = 0:splits−1

range = 1+floor(i*S/splits) : min(S,floor((i+1)*S/splits));
if ~isempty(range)

[X,state.iir] = filter(B,A,double(data(:,range+P)),state.iir,2);
[Xcov,state.cov] = moving_average(N,reshape( ...

bsxfun(@times,reshape(X,1,C,[ ]),reshape(X,C,1,[ ])),C*C,[ ]),state.cov);

update_at = min(1:stepsize:(size(Xcov,2)+stepsize−1),size(Xcov,2));
Xcov = gather(reshape(Xcov(:,update_at),C,C,[ ]));
[last_n,last_R,last_trivial] = deal(0,eye(C),true);
for j = 1:length(update_at)

[V,D] = eig(Xcov(:,:,j));
keep = D<sum((T*V).\hat{ }2) | (1:C)<(C-maxdims);
trivial = all(keep);
if ~trivial

R = real(M*pinv(bsxfun(@times,keep′,V′*M))*V′);

else

R = eye(C);

end
n = update_at(j);
if ~trivial || ~last_trivial

subrange = range((last_n+1):n);
blend = (1-cos(pi*(1:(n-last_n))/(n-last_n)))/2;
data(:,subrange) = bsxfun(@times,blend,R*data(:,subrange)) +

...

bsxfun(@times,1-blend,last_R*data(:,subrange));

end
[last_n,last_R,last_trivial] = deal(n,R,trivial);

end

end

end
state.carry = [state.carry data(:,(end-P+1):end)];
state.carry = state.carry(:,(end-P+1):end);
state.iir = gather(state.iir);
state.cov = gather(state.cov);
data = data(:,1:(end-P));

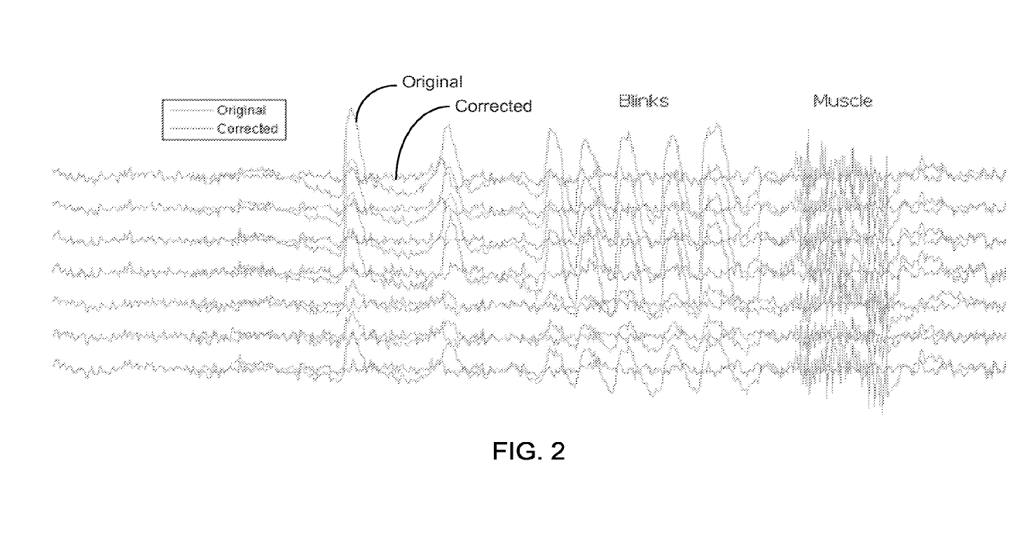

Exemplary implementations of the disclosed method were applied to multi-channel EEG data, and plots of representative data are shown. FIG. 2 shows exemplary results of implementations of the disclosed method in a data plot with the original signal (e.g., labeled and depicted in orange) and a reconstructed signal (e.g., superimposed on the data plot, labeled, and depicted in blue).

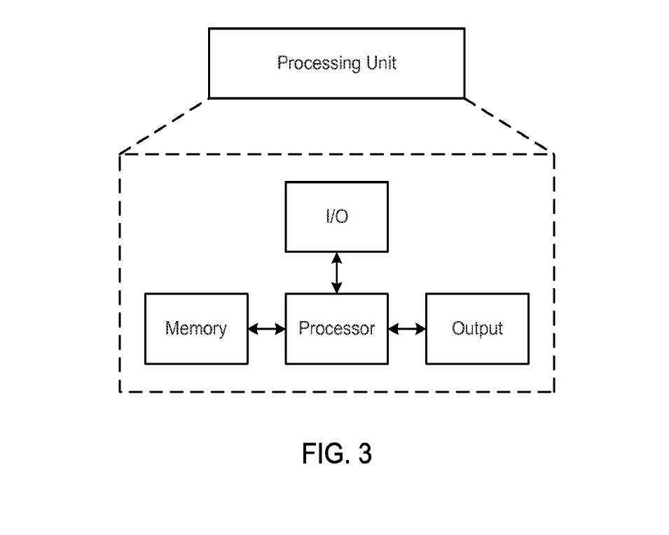

FIG. 3 shows an exemplary computation system for implementing the disclosed methods. FIG. 3 shows one aspect of an exemplary computation or computer system of the disclosed technology capable of implementing the disclosed signal processing methods, in which the computation or computer system includes a processing unit. The processing unit can include a processor to process data that can be in communication with a memory unit to store data. The processing unit can further include an input/output (I/O) unit, and/or an output unit. The exemplary processing unit can be implemented as one of various data processing systems, e.g., such as a personal computer (PC), laptop, tablet, and mobile communication device. To support various functions of the processing unit, the exemplary processor can be included to interface with and control operations of other components of the exemplary processing unit, e.g., including, but not limited to, an I/O unit, an output unit, and a memory unit.

To support various functions of the exemplary processing unit of FIG. 3, the memory unit can store other information and data, such as instructions, software, values, images, and other data processed or referenced by the processor. Various types of Random Access Memory (RAM) devices, Read Only Memory (ROM) devices, Flash Memory devices, and other suitable storage media can be used to implement storage functions of the memory unit. The exemplary memory unit can store data and information. The memory unit can store the data and information that can be used to implement an exemplary signal processing method of the disclosed technology and that can be generated from an exemplary algorithm and model.

To support various functions of the exemplary processing unit of FIG. 3, the I/O unit can be connected to an external interface, source of data storage or processing, and/or display device to receive and transmit data to such interface, storage or processing source, and/or device. For example, various types of wired or wireless interfaces compatible with typical data communication standards, such as Universal Serial Bus (USB), IEEE 1394 (FireWire), Bluetooth, IEEE 802.111, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), and parallel interfaces, can be used to implement the I/O unit. The I/O unit can interface with an external interface, source of data storage, or display device to retrieve and transfer data and information that can be processed by the processor, stored in the memory unit, or exhibited on the output unit.

To support various functions of the exemplary processing unit of FIG. 3, the output unit can be used to exhibit data implemented by the processing unit. The output unit can include various types of display, speaker, or printing interfaces to implement the exemplary output unit. For example, the output unit can include, but is not limited to, a cathode ray tube (CRT), light emitting diode (LED), or liquid crystal display (LCD) monitor or screen as a visual display to implement the output unit. In other examples, the output unit can include, but is not limited to, toner, liquid inkjet, solid ink, dye sublimation, inkless (such as thermal or UV) printing apparatuses to implement the output unit. Also, for example, the output unit can include various types of audio signal transducer apparatuses to implement the output unit. The output unit can exhibit the data and information, as well as store the data and information used to implement an exemplary process of the disclosed technology and from an implemented process.

### Examples

The following examples are illustrative of several embodiments of the present technology. Other exemplary embodiments of the present technology may be presented prior to the following listed examples, or after the following listed examples.

In an example of the present technology (example 1), a method for processing a signal to remove artifacts, comprising includes obtaining a first signal decomposition of a multi-channel baseline signal in a first matrix including nominal non-artifact signal components and a second signal decomposition of a multi-channel data signal in a second matrix including artifact components, in which the first and second matrices are complimentary matrices; forming a linear transform by non-linearly combining the complementary matrices; and producing an output signal corresponding to the multi-channel data signal by applying the formed linear transform to one or more samples of the multi-channel data signal to remove artifacts and retain non-artifact signal content in the output signal.

Example 2 includes the method as in example 1, in which the forming the linear transform by non-linearly combining the two complementary matrices can include classifying signal components of at least one of the multi-channel baseline signal or the multi-channel data signal into artifact and non-artifact components using a binary classification criterion.

Example 3 includes the method as in example 2, in which the binary classification criterion is selected to be sensitive to a quantifiable characteristic of the signal including at least one of amplitude, variance, or a pattern in a time course of the signal.

Example 4 includes the method as in example 2, in which the binary classification criterion can include a data-dependent or empirically estimated parameter or parameters, the parameter or parameters providing a soft or hard threshold to classify the signal components.

Example 5 includes the method as in example 1, in which the multi-channel data signal can include electroencephalograms (EEG), magnetoencephalogram (MEG), electrocorticograms (ECoG), electrocochleograms (ECochG), electrocardiograms (ECG), and/or electromyograms (EMG).

Example 6 includes the method as in example 1, in which the multi-channel data signal can include a single-channel signal augmented to multiple channels, in which at least some channels of the multiple channels having a replicate of the single-channel signal that is temporally modified.

Example 7 includes the method as in example 1, in which the multi-channel baseline signal can include a calibration signal corresponding to the multi-channel data signal or a predetermined signal.

Example 8 includes the method as in example 1, in which the multi-channel baseline signal can include at least a portion of the multi-channel data signal.

Example 9 includes the method as in example 1, in which the first signal decomposition can include a component representation of the multi-channel baseline signal.

Example 10 includes the method as in example 1, in which the second signal decomposition can include a component representation of the multi-channel data signal.

Example 11 includes the method as in example 1, in which the second signal decomposition can include a component representation of the multi-channel baseline signal.

Example 12 includes the method as in example 1, in which the second signal decomposition of the multi-channel data signal can include a signal decomposition or a component representation of a segment of the multi-channel data signal.

Example 13 includes the method as in example 1, in which the first matrix primarily can include the nominal non-artifact signal components.

Example 14 includes the method as in example 1, in which one or both of the first matrix and the second matrix can include eigenvectors of a principal component analysis.

Example 15 includes the method as in example 1, in which the second matrix can include eigenvectors of a principal component analysis of a short signal segment or resulting from an alternative decomposition of the short signal segment capable of isolating high-amplitude components, and in which the non-linearly combining the two complementary matrices can include solving an underdetermined linear system.

Example 16 includes the method as in example 1, in which the method can further include estimating the second matrix from data containing low artifact content, the estimating including making an explicit or an implicit assumption of low artifact content.

In another example of the present technology (example 17), a computer program product comprising a computer-readable storage medium having code stored thereon, the code, when executed, causing a processor of a computer or computer system in a communication network to implement a method for processing a signal to remove artifacts, in which the computer program product is operated by the computer or computer system to implement the method comprising: obtaining a first signal decomposition of a multi-channel baseline signal in a first matrix including nominal non-artifact signal components and a second signal decomposition of a multi-channel data signal in a second matrix including artifact components, in which the first and second matrices are complimentary matrices; forming a linear transform by non-linearly combining the complementary matrices; and producing an output signal corresponding to the multi-channel data signal by applying the formed linear transform to one or more samples of the multi-channel data signal to remove artifacts and retain non-artifact signal content in the output signal.

Example 18 includes the computer program product as in example 17, in which the forming the linear transform by non-linearly combining the two complementary matrices can include classifying signal components of at least one of the multi-channel baseline signal or the multi-channel data signal into artifact and non-artifact components using a binary classification criterion.

Example 19 includes the computer program product as in example 18, in which the binary classification criterion is selected to be sensitive to a quantifiable characteristic of the signal including at least one of amplitude, variance, or a pattern in a time course of the signal.

Example 20 includes the computer program product as in example 18, in which the binary classification criterion can include a data-dependent or empirically estimated parameter or parameters, the parameter or parameters providing a soft or hard threshold to classify the signal components.

Example 21 includes the computer program product as in example 17, in which the multi-channel data signal can include electroencephalograms (EEG), magnetoencephalogram (MEG), electrocorticograms (ECoG), electrocochleograms (ECochG), electrocardiograms (ECG), and/or electromyograms (EMG).

Example 22 includes the computer program product as in example 17, in which the multi-channel data signal can include a single-channel signal augmented to multiple channels, in which at least some channels of the multiple channels having a replicate of the single-channel signal that is temporally modified.

In another example of the present technology (example 23), a method for removing artifacts from an electrophysiological signal includes producing a first signal decomposition or component representation of a multi-channel baseline signal in a first matrix and a second signal decomposition or component decomposition of a measured multi-channel data signal of an electrophysiological signal from a subject, in which the first matrix includes nominal non-artifact signal components and the second matrix includes artifact components, and the first matrix and the second matrix are complimentary matrices; forming a linear transform by non-linearly combining the complementary matrices including classifying signal components of one or both of the first matrix and second matrix into artifact and non-artifact components using a binary classification criterion; and producing an output signal corresponding to the electrophysiological signal by applying the formed linear transform to one or more samples of the measured multi-channel data signal to remove amplitude artifacts and retain non-artifact signal content in the output signal.

Example 24 includes the method as in example 23, in which the binary classification criterion is selected to be sensitive to a quantifiable characteristic of the signal including at least one of amplitude, variance, or a pattern in a time course of the signal.

Example 25 includes the method as in example 23, in which the electrophysiological signal can include electroencephalograms (EEG), electrocorticograms (ECoG), electrocochleograms (ECochG), electrocardiograms (ECG), and/or electromyograms (EMG).

Example 26 includes the method as in example 25, in which the multi-channel baseline signal can include an EEG calibration signal recorded when the subject remains stationary.

In another example of the present technology (example 27), a method for removal of artifacts in multi-channel signals includes forming a linear transform by non-linearly combining two complementary matrices of signal components of a data signal and a baseline signal, a first matrix containing non-artifact signal components of the baseline signal and a second matrix including artifact components of the data signal; and applying the formed linear transform to one or more samples of the data signal to reduce artifacts in the one or more samples to which the linear transform is applied, in which the applying includes retaining residual non-artifact signal content in the signal components.

Example 28 includes the method as in example 27, in which the first matrix primarily includes the non-artifact signal components.

Example 29 includes the method as in example 27, in which the second matrix is composed of eigenvectors of a principal component analysis of a short signal segment of the data signal or resulting from an alternative decomposition of the signal segment capable of isolating high-amplitude components.

Example 30 includes the method as in example 29, in which the non-linearly combining the two complementary matrices can include solving an underdetermined linear system.

Example 31 includes the method as in example 27, in which the method can further include estimating the second matrix from data containing low artifact content, the estimating including making an explicit or an implicit assumption of low artifact content.

Example 32 includes the method as in example 27, in which the non-linearly combining the two complementary matrices can include classifying signal components into artifact and non-artifact components.

Example 33 includes the method as in example 32, in which the classifying the signal components can include using a classification criterion that is substantially sensitive to a quantifiable characteristic of the signal including at least one of amplitude, variance, or a pattern in a time course of the signal.

Example 34 includes the method as in example 32, in which the classification criterion can include a data-dependent or empirically estimated parameter or parameters, the parameter or parameters serving a role as a soft or hard threshold.

Exemplary Implementations Using a Wearable EEG System

In another aspect of the disclosed technology, methods, systems, and devices are disclosed for real-time modeling and 3D visualization of source dynamics and connectivity using wearable EEG systems.

Implementations of the disclosed systems and devices can deliver real-time data extraction, preprocessing, artifact rejection, source reconstruction, multivariate dynamical system analysis (including spectral Granger causality) and 3D visualization as well as classification within the open-source SIFT and BCILAB toolboxes.

Dynamic cortico-cortical interactions are central to neuronal information processing. The ability to monitor these interactions in real time may prove useful for Brain-Computer Interface (BCI) and other applications, providing information not obtainable from univariate measures, e.g., such as bandpower and evoked potentials. Wearable (mobile, unobtrusive) EEG systems likewise play an important role in BCI applications, affording data collection in a wider range of environments. However, reliable real-time modeling of neuronal source dynamics using data collected in mobile settings faces challenges, including mitigating artifacts and maintaining fast computation and good modeling performance with limited amount of data.

Described below are some of exemplary wearable hardware and signal processing methods capable of addressing these challenges, contributing to the development of EEG as a mobile brain imaging modality. Exemplary implementations of the disclosed real-time modeling and 3D visualization technology were performed, showing that the application of the disclosed methods can provide a pipeline to simulated data and real EEG data obtained from a novel wearable high-density (64-channel) dry EEG system.

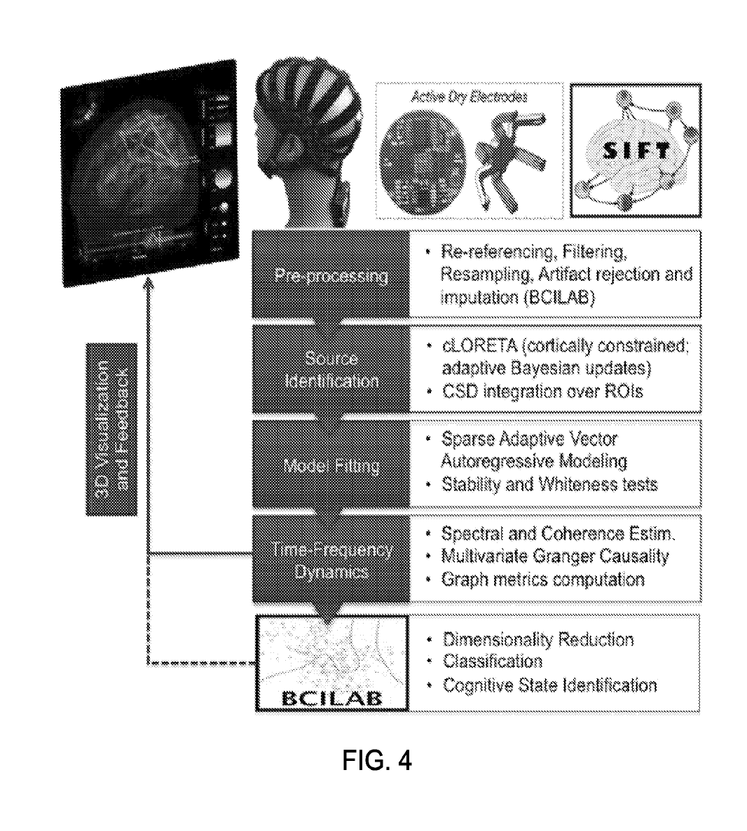

The exemplary implementations included the following materials and methods. FIG. 4 shows a diagram of an exemplary system of the disclosed technology including a real-time data processing pipeline. The exemplary system includes an exemplary 64-channel EEG system with flexible active dry electrodes. A padded sensor is used on the forehead locations for contact with bare skin. For through-hair recordings, a novel dry electrode is used, including a flexible plastic substrate coated with a conductive surface. The legs of the sensor push through hair to achieve contact with scalp, flattening with pressure to minimize discomfort and injury. For example, typical contact impedances were 100 kΩ-1 MΩ, and high input impedance amplifiers were used to ensure minimal signal degradation. For example, typical correlation between simultaneously recorded averaged evoked potentials (e.g., AEP, SSVEP, P300) for dry and standard wet electrodes is r>0.9 indicating comparable signal measurement. Nonetheless, the dry electrodes may exhibit a higher level of drift and low frequency noise due to the lack of gel and skin-abrasion.

In implementations using the exemplary EEG system, all electronics, e.g., including preamplifiers, digitization, battery (6-7 hour capacity), onboard impedance monitoring, micro-controller and Bluetooth transceiver were contained in a miniature box at the rear of the headset. Signals are sampled at 300 Hz with 24-bit precision. The total noise of the data acquisition circuitry, within EEG bandwidth, was less than 1 μV RMS. Event markers were transmitted from the PC to the headset via a special low-latency wireless link specifically optimized to minimize jitter (<500 microseconds).

The exemplary implementations included pre-processing and artifact rejection techniques. EEG data were streamed into MATLAB (The Mathworks, Natick, Mass.), and an online-capable pre-processing pipeline was applied using BCILAB. Elements of this pipeline may be initialized on a short segment of calibration data. These include rejection (and optional re-interpolation) of corrupted data samples or channels. In some implementations, for example, short-time high-amplitude artifacts in the continuous data may be removed online, using the disclosed methods (e.g., depicted in FIGS. 1A and 1B), which are referred to as Artifact Subspace Reconstruction (ASR). For example, the implementations of the disclosed ASR techniques included using a sliding-window Principal Component Analysis, which statistically interpolates any high-variance signal components exceeding a threshold relative to the covariance of the calibration dataset. Each affected time point of EEG was then linearly reconstructed from the retained signal subspace based on the correlation structure observed in the calibration data. In some implementations of the wearable EEG systems, for example, artifacts may also be removed by rejecting a subspace of ICA components pre-computed using an (overcomplete) decomposition on calibration data or adaptively estimated using Online Recursive ICA. Artifactual components may be identified automatically by fitting dipoles to components and selecting a subspace of components to retain based on heuristic criteria such as residual variance of dipole fit or dipole anatomical coordinates and labels.

The exemplary implementations included source reconstruction techniques. For example, following pre-processing, one may estimate the primary current source density (CSD) over a medium- to high-resolution cortical mesh (e.g., 3751-12000 vertices). In the exemplary implementations, a 3751-vertex mesh was used. The default forward model included a four-layer (e.g., skull, scalp, csf, and cortex) Boundary Element Method (BEM) model derived from the MNI Colin 27 brain and computed using OpenMEEG. For inverse modeling, anatomically constrained LORETA with Bayesian hyperparameter estimation was relied upon. This approach can be well suited for real-time adaptive estimation and automatically controls the level of regularization for each measurement vector. The SVD-based reformulation was followed for processing speed. Additionally, segmented were the source space into 90 regions of interest (ROIs) using Automated Anatomical Labeling (AAL). The user can compute spatially averaged, integrated or maximal CSD for any subset of these ROIs. Routines from an exemplary MoBILAB toolbox freely available online were used.

The exemplary implementations included dynamic system analysis. Preprocessed channel or source time-series were forwarded to SIFT and an order-p sparse vector autoregressive (VAR[p]) model was fit to a short chunk of recent data (e.g., 0.5-2 sec). The VAR coefficients were estimated using Alternating Direction Method of Multipliers (ADMM) with a Group Lasso penalty. Model estimation was warm-started using the solution for the previous data chunk. The regularization parameter can be initialized by cross-validation on the calibration data and/or adapted online using a heuristic based on two-point estimates of the gradients of the primal and dual norms. Model order can be selected offline, by minimizing information criteria (e.g. AIC or BIC) on calibration data. Following model fitting and tests of stability and residual whiteness (autocorrelation function or Portmanteau), obtained were the spectral density matrix and any of the frequency-domain functional and effective connectivity measures implemented in SIFT. Graph-reductive metrics such as degree, flow, and asymmetry ratio can be applied to connectivity matrices. These measures may be piped to BCILAB as features for subsequent classification or visualized in real time. Available graphs include current density, power spectra, connectivity, outflow, etc., in 2-D plots as well interactively within a three-dimensional model of the head and brain.

The exemplary implementations included connectivity classification. To learn robust predictive models on the high-dimensional connectivity feature space (d>7000) from few trials, strong prior assumptions need to be employed, for example. Applied was a regularized logistic regression, implemented via ADMM, to log-transformed time/frequency (T/F) Direct Directed Transfer Function (dDTF) measures (yielding a 4-dimensional feature tensor across pairwise connectivity, time and frequency). The regularization simultaneously employed a sparsifying l₁/l₂+l₁ norm with one group for each connectivity edge, containing its associated T/F weights, plus three trace norm terms to couple the T/F weights for all out-edges of a node, all in-edges of a node, and all auto-edges across nodes, respectively, plus an l₂ smoothness term across time and frequency, respectively. The regularization parameter for the data term was searched via (nested) 5-fold blockwise cross-validation over the range 2⁰, 2^{-0.25}, . . . , 2⁻¹⁰. The relative weights of the regularization terms were searched over 10 predefined joint assignments, although setting all weights simply to 1 yielded comparable results.

The exemplary implementations included collecting exemplary data using the described pipeline on both simulated data and real (e.g., 64-channel) data collected in mobile settings using the EEG hardware (shown in FIG. 4).

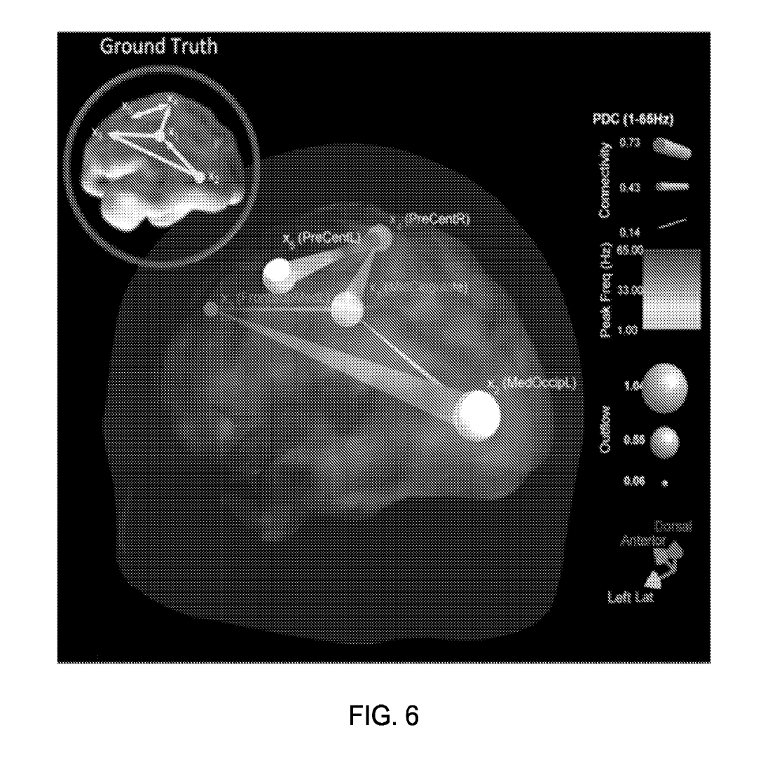

1) Simulated Data: To test the ability of our pipeline to accurately reconstruct source dynamics and connectivity in real-time, generated was a five-dimensional VAR[3] system of coupled oscillators. This comprised the CSD time-series of 5 sources positioned on a 3571-vertex cortical mesh. For example, each source had a Gaussian spatial distribution (6=5 cm) with mean equal to the centroid of each of the following AAL ROIs (respectively): x₁: Left Middle Cingulate Gyms, x₂: Left Middle Occipital Gyms, x₃: Right Medial Superior Frontal Gyms, x₄: Right Precentral Gyms, x₅: Left Precentral Gyms. This exemplary system is depicted in the inset in FIG. 6. Generated were two minutes of source time-series data (Fs=300 Hz) and projected this through the realistic forward model described in Section II-C to produce 64-channel EEG data. Gaussian i.i.d sensor noise was added (SNR=σ_{data}/σₙₒᵢₛₑ=5).

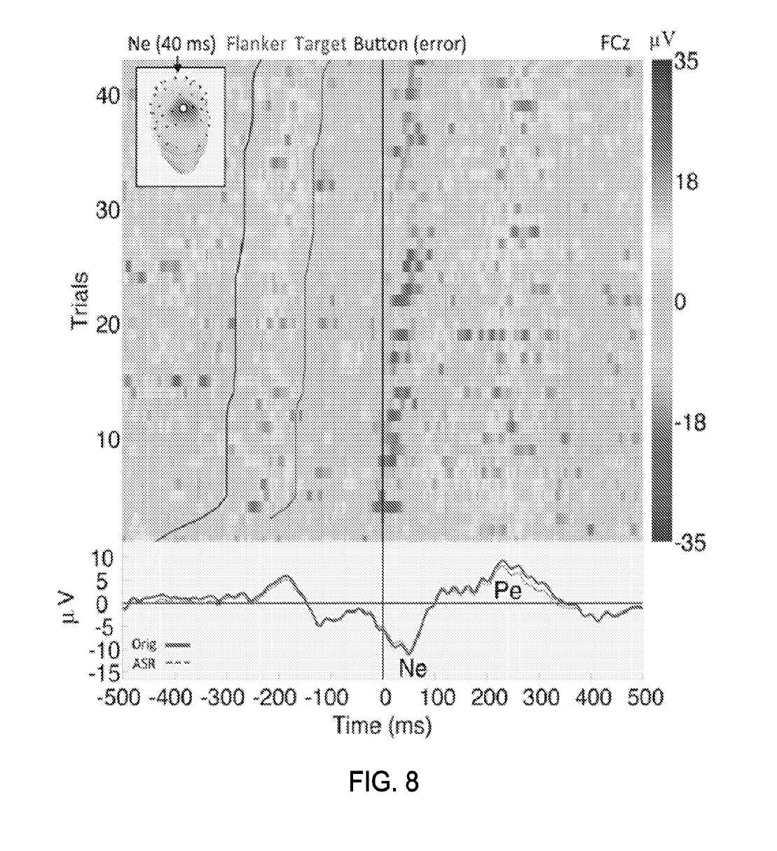

2) Real Data: One session of EEG data was collected from a 22 year-old right-handed male performing a modified Eriksen Flanker task with a 133 ms delay between flanker and target presentation. Flanker tasks have been extensively studied and are known to produce error-related negativity (ERN, Ne) and error-related positivity (P300, Pe) event-related potentials (ERPs) following error commission, as shown on the bottom portion of FIG. 8. The Ne is an early negativity in the EEG with middle/anterior cingulate generators which peaks 40-80 ms following commission of a response error. This is often followed by a Pe peaking at 200-500 ms.

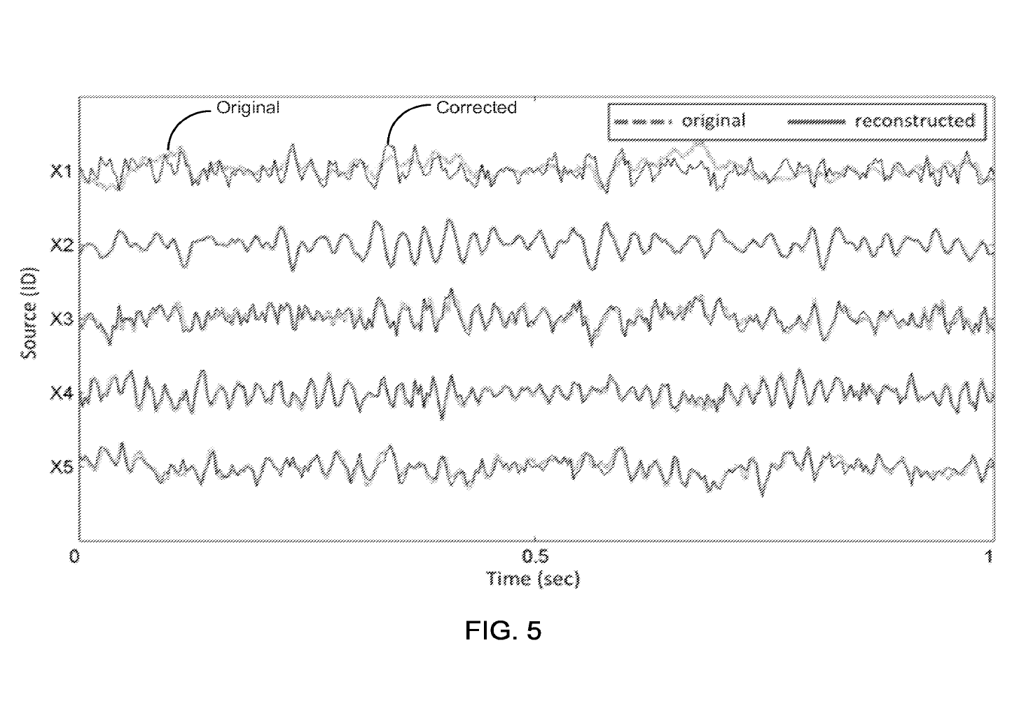

Exemplary results of the exemplary implementations with the EEG system included the following. Exemplary Simulated Data. The simulated EEG data was piped through the exemplary pre-processing pipeline (e.g., in which filtering and ASR disabled here) and median CSD was computed for each of the 5 ROIs. FIG. 5 shows a data plot depicting exemplary results of exemplary implementations of the disclosed technology. The data plot of FIG. 5 depicts exemplary signal data of original (e.g., red, dashed line) vs. reconstructed (e.g., blue, solid line) current source density for a 1-sec segment of our 5 simulated ROIs. Sources and data are scaled to unit variance in the exemplary data plot. For example, a 1-sec segment of reconstructed CSD superimposed on the true CSD. Superficial sources were accurately recovered, while the deep, tangential source (X₁; mid-cingulum) was somewhat more poorly reconstructed. Spectral density, dDTF and partial directed coherence (PDC) between ROI median-CSD time-series was estimated using a 1-sec sliding window over 1-65 Hz. The max operator was applied to collapse across frequency producing a 2D connectivity matrix (directed graph). FIG. 6 shows a diagram depicting an exemplary temporal snapshot (of a representative time window) of reconstructed source networks (e.g., PDC estimator), e.g., displayed within a BrainMovie3D visualizer. As shown in the diagram of FIG. 6, the edge color denotes preferred coupling frequency while edge size and tapering, respectively, denote coupling strength and directionality at that frequency. Node size indicates outflow (net influence of a source on all other sources). The graph shown in the diagram of FIG. 6 depicts the thresholded at the commonly used PDC heuristic level of 0.1. Cortical surface regions are colored according to their AAL atlas label (90 regions). Ground truth is displayed in the inset of FIG. 6. Over all time windows, the connectivity graph was recovered with high accuracy—the average area under curve (AUC) was 0.97+/−0.021. Peak coupling frequency and relative strength were also correctly recovered.

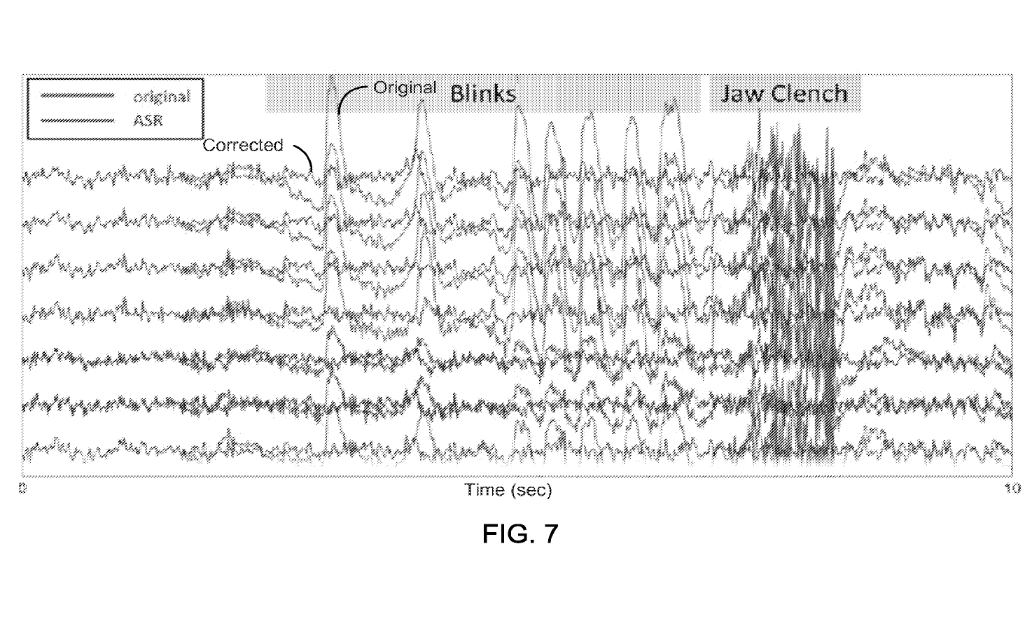

Exemplary Real Data. 1) Data Quality and Artifact Rejection: In these exemplary implementations, the online pipeline included the following pre-processing elements (in order of application), for example: downsampling to 128 Hz, sub-1 Hz drift correction (Butterworth IIR filter), bad channel removal and interpolation, ASR, average referencing and 50 Hz low-pass minimum-phase FIR filtering. Four channels were automatically removed (e.g., Afz, T7, T8, P09), which were those also identified by eye as corrupted during data collection. FIG. 7 shows a data plot depicting exemplary results of exemplary implementations of the disclosed technology. The exemplary data plot of FIG. 7 shows 10 sec of EEG data following ASR data cleaning (e.g., blue trace) superimposed on original data (e.g., depicted by the red trace). As shown in the data plot, there are segments of EEG data contaminated by blink and muscle artifacts, e.g., before and after ASR artifact removal.

FIG. 8 shows an ERP image depicting single-trial EEG potentials (e.g., no smoothing) at FCz for response-locked error trials, sorted by latency of response to target onset (red sigmoidal trace). Responses occurred at 0 ms (vertical line). The bottom panel shows the averaged ERP without ASR in blue, and the ERP with ASR enabled in red. The single-trial EEG data for response-locked error trials are shown for electrode FCz in FIG. 8. The exemplary trials are sorted by reaction time. Although acausal filters cannot be used online, for this plot alone, in order to accurately assess ERP latencies, all filters were zero phase (acausal). The analysis was run with and without ASR (the latter shown here) and confirmed that ASR did not distort ERPs (FIG. 8, red trace). Note that nearly every trial shows a visual evoked response to the stimulus as well as prominent Ne and Pe following the erroneous button press. The scalp topography of the Ne (upper left) has a frontocentral distribution centered at FC_{z}, as expected for a mid/anterior cingulate or frontal midline generator. Encouragingly, the quality of the evoked responses was comparable to that reported using research-grade gel-based EEG systems.

2) Source Reconstruction: The exemplary source analysis showed good reconstruction of early visual evoked potentials from occipital and parietal regions as well as frontal localization of the Pe following button presses. However, the Ne was not reliably recovered in these exemplary implementations. As noted above, single-trial estimates of current density may be less reliable for deep, medial sources (e.g., cingulate regions) than for superficial sources.

3) Connectivity Analysis: For a moderate number of channels (or sources) (e.g., 8-15) with model order in the 10-15 range, for example, generally obtained were good VAR model fit (e.g., stable with uncorrelated residuals, ACF test: p<0.05). The VAR process spectrum exhibits characteristic EEG 1/f shape with theta, alpha, and beta peaks, including prominent occipital alpha gain and occipital-frontal Granger-causality at rest with eyes closed.

4) Classification: The exemplary classification stage was tested on the problem of detecting human response error commission from EEG data for which univariate source processes, e.g., such as event-related potentials (ERPs), have been employed in the past. However, for example, effective connectivity features have not been used in this context. To this end, for example, dDTF estimates were used between channels FP1, FP2, FCz, C3, C4, PO3 and PO4 selected after 64-channel data cleaning. Analysis was performed on epochs at −0.5 to 1.5 seconds relative to button press events and time-frequency dDTF was computed in a 1-sec sliding window within each epoch. A 5-fold blockwise cross-validation was performed with clean separation of testing and training data to measure AUC on 172 trials, yielding a mean AUC of 0.895+/−0.047. In order to compare with more conventional features, also performed was a 5-fold classification using a state-of-the-art first-order ERP method. Obtained was a mean AUC of 0.97+/−0.02. Given the saliency of error-related ERP features in this dataset, it is not surprising that the ERP-based method performed extremely well, outperforming higher-dimensional connectivity features.

The presented EEG system of the disclosed technology is capable of real-time analysis. For example, on a suitable processing unit (e.g., such as an Intel i7 4-core (2.3 Ghz) laptop), preprocessing and source reconstruction (e.g., 3751 vertex mesh) typically takes 50-80 ms, model fitting 50-70 ms, classification under 5 ms and (optional) MATLAB-based visualization 200-300 ms. The channel connectivity features can contain information relevant to error detection, and the disclosed system can be implemented to examine source connectivity as well attempt prediction of error commission from pre-stimulus dynamics where time-domain ERP features are absent.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A method for processing a signal to remove artifacts, comprising:
obtaining a first signal decomposition of a multi-channel baseline signal in a first matrix including nominal non-artifact signal components and a second signal decomposition of a multi-channel data signal in a second matrix including artifact components, wherein the first and second matrices are complimentary matrices;
forming a linear transform by non-linearly combining the complementary matrices; and
producing an output signal corresponding to the multi-channel data signal by applying the formed linear transform to one or more samples of the multi-channel data signal to remove artifacts and retain non-artifact signal content in the output signal.

2. The method as in claim 1, wherein the forming the linear transform by non-linearly combining the two complementary matrices includes classifying signal components of at least one of the multi-channel baseline signal or the multi-channel data signal into artifact and non-artifact components using a binary classification criterion.

3. The method as in claim 2, wherein the binary classification criterion is selected to be sensitive to a quantifiable characteristic of the signal including at least one of amplitude, variance, or a pattern in a time course of the signal.

4. The method as in claim 2, wherein the binary classification criterion includes a data-dependent or empirically estimated parameter or parameters, the parameter or parameters providing a soft or hard threshold to classify the signal components.

5. The method as in claim 1, wherein the multi-channel data signal includes at least one of electroencephalograms (EEG), magnetoencephalogram (MEG), electrocorticograms (ECoG), electrocochleograms (ECochG), electrocardiograms (ECG), or electromyograms (EMG).

6. The method as in claim 1, wherein the multi-channel data signal includes a single-channel signal augmented to multiple channels, wherein at least some channels of the multiple channels having a replicate of the single-channel signal that is temporally modified.

7. The method as in claim 1, wherein the multi-channel baseline signal includes a calibration signal corresponding to the multi-channel data signal or a predetermined signal.

8. The method as in claim 1, wherein the multi-channel baseline signal comprises at least a portion of the multi-channel data signal.

9. The method as in claim 1, wherein the first signal decomposition includes a component representation of the multi-channel baseline signal.

10. The method as in claim 1, wherein the second signal decomposition includes a component representation of the multi-channel data signal.

11. The method as in claim 1, wherein the second signal decomposition includes a component representation of the multi-channel baseline signal.

12. The method as in claim 1, wherein the second signal decomposition of the multi-channel data signal includes a signal decomposition or a component representation of a segment of the multi-channel data signal.

13. The method as in claim 1, wherein the first matrix primarily includes the nominal non-artifact signal components.

14. The method as in claim 1, wherein one or both of the first matrix and the second matrix comprises eigenvectors of a principal component analysis.

15. The method as in claim 1, wherein the second matrix comprises eigenvectors of a principal component analysis of a short signal segment or resulting from an alternative decomposition of the short signal segment capable of isolating high-amplitude components, and wherein the non-linearly combining the two complementary matrices includes solving an underdetermined linear system.

16. The method as in claim 1, further comprising:
estimating the second matrix from data containing low artifact content, the estimating including making an explicit or an implicit assumption of low artifact content.

17. A computer program product comprising a computer-readable storage medium having code stored thereon, the code, when executed, causing a processor of a computer or computer system in a communication network to implement a method for processing a signal to remove artifacts, wherein the computer program product is operated by the computer or computer system to implement the method comprising:
obtaining a first signal decomposition of a multi-channel baseline signal in a first matrix including nominal non-artifact signal components and a second signal decomposition of a multi-channel data signal in a second matrix including artifact components, wherein the first and second matrices are complimentary matrices;
forming a linear transform by non-linearly combining the complementary matrices; and
producing an output signal corresponding to the multi-channel data signal by applying the formed linear transform to one or more samples of the multi-channel data signal to remove artifacts and retain non-artifact signal content in the output signal.

18. The computer program product as in claim 17, wherein the forming the linear transform by non-linearly combining the two complementary matrices includes classifying signal components of at least one of the multi-channel baseline signal or the multi-channel data signal into artifact and non-artifact components using a binary classification criterion.

19. The computer program product as in claim 18, wherein the binary classification criterion is selected to be sensitive to a quantifiable characteristic of the signal including at least one of amplitude, variance, or a pattern in a time course of the signal.

20. The computer program product as in claim 18, wherein the binary classification criterion includes a data-dependent or empirically estimated parameter or parameters, the parameter or parameters providing a soft or hard threshold to classify the signal components.

21. The computer program product as in claim 17, wherein the multi-channel data signal includes at least one of electroencephalograms (EEG), magnetoencephalogram (MEG), electrocorticograms (ECoG), electrocochleograms (ECochG), electrocardiograms (ECG), or electromyograms (EMG).

22. The computer program product as in claim 17, wherein the multi-channel data signal includes a single-channel signal augmented to multiple channels, wherein at least some channels of the multiple channels having a replicate of the single-channel signal that is temporally modified.

23. A method for removing artifacts from an electrophysiological signal, comprising:
producing a first signal decomposition or component representation of a multi-channel baseline signal in a first matrix and a second signal decomposition or component decomposition of a measured multi-channel data signal of an electrophysiological signal from a subject, wherein the first matrix includes nominal non-artifact signal components and the second matrix includes artifact components, and the first matrix and the second matrix are complimentary matrices;
forming a linear transform by non-linearly combining the complementary matrices including classifying signal components of one or both of the first matrix and second matrix into artifact and non-artifact components using a binary classification criterion; and
producing an output signal corresponding to the electrophysiological signal by applying the formed linear transform to one or more samples of the measured multi-channel data signal to remove amplitude artifacts and retain non-artifact signal content in the output signal.

24. The method as in claim 23, wherein the binary classification criterion is selected to be sensitive to a quantifiable characteristic of the signal including at least one of amplitude, variance, or a pattern in a time course of the signal.

25. The method as in claim 23, wherein the electrophysiological signal includes at least one of electroencephalograms (EEG), electrocorticograms (ECoG), electrocochleograms (ECochG), electrocardiograms (ECG), or electromyograms (EMG).

26. The method as in claim 25, wherein the multi-channel baseline signal includes an EEG calibration signal recorded when the subject remains stationary.

27. A method for removal of artifacts in multi-channel signals, comprising:
forming a linear transform by non-linearly combining two complementary matrices of signal components of a data signal and a baseline signal, a first matrix containing non-artifact signal components of the baseline signal and a second matrix including artifact components of the data signal; and
applying the formed linear transform to one or more samples of the data signal to reduce artifacts in the one or more samples to which the linear transform is applied, wherein the applying includes retaining residual non-artifact signal content in the signal components.

28. The method as in claim 27, wherein the first matrix primarily includes the non-artifact signal components.

29. The method as in claim 27, wherein the second matrix is composed of eigenvectors of a principal component analysis of a short signal segment of the data signal or resulting from an alternative decomposition of the signal segment capable of isolating high-amplitude components.

30. The method as in claim 29, wherein the non-linearly combining the two complementary matrices includes solving an underdetermined linear system.

31. The method as in claim 27, further comprising:
estimating the second matrix from data containing low artifact content, the estimating including making an explicit or an implicit assumption of low artifact content.

32. The method as in claim 27, wherein the non-linearly combining the two complementary matrices includes classifying signal components into artifact and non-artifact components.

33. The method as in claim 32, wherein the classifying the signal components includes using a classification criterion that is substantially sensitive to a quantifiable characteristic of the signal including at least one of amplitude, variance, or a pattern in a time course of the signal.

34. The method as in claim 32, wherein the classification criterion includes a data-dependent or empirically estimated parameter or parameters, the parameter or parameters serving a role as a soft or hard threshold.
